(19) United States
(12) Patent Application Publication
(10) Pub. No.: US 2023/0036148 A1
(43) Pub. Date: Feb. 2, 2023

(54) DRESSING WITH OPTIONAL WOUND STATUS INDICATOR

(71) Applicant: KCI Licensing, Inc., San Antonio, TX (US)

(72) Inventor: Christopher Brian LOCKE, Bournemouth (GB)

(21) Appl. No.: 17/790,922

(22) PCT Filed: Jan. 7, 2021
(86) PCT No.: PCT/IB2021/050100

§ 371 Date: Jul. 5, 2022

Related U.S. Application Data
(60) Provisional application No. 62/958,471, filed on Jan. 8, 2020.

(51) Int. Cl. A61F 13/00; A61F 13/02; A61M 1/00

(52) U.S. Cl. CPC A61F 13/00055; A61F 13/0223; A61F 13/0206; A61F 13/025; A61F 13/0216; A61M 1/912; A61F 13/0266; A61F 13/00076; A61M 1/915

(57) ABSTRACT

A dressing for a wound includes a drape, an absorbent core, and a wicking member. The drape can be configured to sealingly couple with a patient's skin surrounding the wound. The drape may enclose an inner volume of the dressing. The absorbent core layer may be positioned within the inner volume of the dressing. The wicking member is positioned within the inner volume, wherein the wicking member extends from an underside of the absorbent core layer to the drape. The indicator may be configured to fluidly couple with the wicking member through the drape and receive fluid from the wicking member. The indicator may be configured to provide a visual wound status indication in response to receiving fluid from the wound through the wicking member.

### CROSS-REFERENCE TO RELATED APPLICATIONS

This application claims the benefit of priority to U.S. Provisional Application No. 62/958,471, filed on Jan. 8, 2020, which is incorporated herein by reference in its entirety.

### BACKGROUND

The present disclosure relates generally to wound dressings. Wound dressings can provide a sterile interface to guard a patient's wound from contamination during the healing process. Dressings may also absorb wound fluid and/or increase the rate that wound fluid is removed from a wound bed. During the healing process, wound fluid can build up within the dressing and/or within an absorbent article therein. It would be desirable to provide a single wound dressing having an optional wound status indicator adaptable for use in applications where status indication is desired and where status indication may not be deemed necessary.

### SUMMARY

One implementation of the present disclosure is a dressing for a wound, according to some embodiments. In some embodiments, the dressing includes a drape, an absorbent core, and a wicking member. The drape can be configured to sealingly couple with a patient's skin surrounding the wound. In some embodiments, the drape encloses an inner volume of the dressing. In some embodiments, the absorbent core layer is positioned within the inner volume of the dressing. In some embodiments, the wicking member is positioned within the inner volume. In some embodiments, the wicking member extends from an underside of the absorbent core layer to the drape. In some embodiments, the indicator is configured to fluidly couple with the wicking member through the drape and receive fluid from the wicking member. In some embodiments, the indicator is configured to provide a visual wound status indication in response to receiving fluid from the wound through the wicking member.

Another implementation of the present disclosure is a convertible dressing for a wound. In some embodiments, the convertible dressing includes an absorbent core, a wicking member, and a drape. The wicking member may extend along an exterior surface of the absorbent core. The drape can be configured to extend over the absorbent core and the wicking member. The drape can include a removable portion configured to expose or produce an aperture in the drape. The aperture may be aligned with the wicking member.

Another implementation of the present disclosure is a method of providing a dressing with a wound status indication. The method can include providing a dressing having a wound drape that seals with a user's skin surrounding a wound. The dressing may include an absorbent core and a wicking member within the wound drape. The wicking member can extend between an underside of the absorbent core and the wound drape. The method can include providing an opening in the wound drape aligned with the wicking member. The method can also include providing a visual indicator at the opening. The visual indicator is configured to provide a visual wound status indication in response to receiving fluid from the wicking member.

Another implementation of the present disclosure is a kit for a wound dressing with optional status indication. The kit may include a sealed package, a wound dressing, and one or more wound status indicators. The sealed package may have a sterilized interior containing at least the wound dressing and the one or more wound status indicators. The wound dressing may include a drape, an absorbent core, and a wicking layer. The drape may include an opening and a removable cover on the opening. The one or more wound status indicators may be configured to be optionally placed over the opening in communication with the wicking layer after removal of the removable cover.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a top view of a dressing with an optional wound status indicator, according to some embodiments.

FIG. 2 is a perspective view of the dressing with the optional wound status indicator of FIG. 1, according to some embodiments.

FIG. 3 is a side sectional view of the dressing of FIG. 1, according to some embodiments.

FIG. 4 is a side sectional view of the dressing of FIG. 1, showing a fluid flow path, according to some embodiments.

FIG. 5 is a flow diagram of a process for using the dressing of FIG. 1, according to some embodiments.

FIG. 6 is a block diagram of a wound therapy system including a therapy device coupled to the wound dressing of FIGS. 1-4 via tubing, according to some embodiments.

FIG. 7 is a block diagram illustrating the therapy device of FIG. 6 in greater detail when the therapy device operates to draw a vacuum within a negative pressure circuit, according to some embodiments.

FIG. 8A is a block diagram illustrating the therapy device of FIG. 6 in greater detail when the therapy device operates to vent the negative pressure circuit, according to some embodiments.

FIG. 8B is a block diagram illustrating the therapy device of FIG. 6 in greater detail when the therapy device uses an orifice to vent the negative pressure circuit, according to some embodiments.

FIG. 9 is a block diagram illustrating the therapy device of FIG. 6 in greater detail when the therapy device operates to deliver instillation fluid to the wound dressing and/or a wound, according to some embodiments.

FIG. 10 is a side sectional view of the dressing of FIG. 1 showing an alternative configuration of a wicking member, according to some embodiments.

FIG. 11 is a side sectional view of the dressing of FIG. 1 showing an alternative configuration of the wicking member, according to some embodiments.

### DETAILED DESCRIPTION

### Overview

Referring generally to the FIGURES, a wound dressing includes a drape layer, an absorbent core, and a wicking structure. The drape layer may be configured to sealingly couple with patient's skin surrounding a wound to define an inner or internal volume of the wound dressing. In some embodiments, the drape layer includes an adhesive that is coated along at least a portion of a periphery of an interior or wound-facing surface of the drape layer.

The absorbent core and the wicking structure are positioned within the drape layer. In some embodiments, the absorbent core and the wicking structure are positioned between the patient's skin/wound and the interior surface of the drape layer. The absorbent core may abut, directly engage, contact, be placed over, be positioned over, etc., the patient's wound. The wicking structure may be a strip or an elongated thin member that extends between the drape layer and the absorbent core. The wicking structure may extend laterally along an outwards or exterior surface of the absorbent core. In some embodiments, the wicking structure extends at least partially underneath the absorbent core or between the absorbent core and the patient's skin/wound. The wicking structure may fluidly couple at one end with the patient's wound so that the wicking structure absorbs fluid exuded by the wound. The wicking structure can extend to an opposite side of the absorbent structure between the drape layer and the absorbent core.

In some embodiments, the drape layer includes an opening, a hole, an aperture, a port, a window, etc., that extends through an outwards side of the drape layer. The opening may be aligned with the wicking structure to facilitate a flow path from the wound, along the wicking structure, and out of the opening. In some embodiments, the opening is covered with a cover layer that can be removed by a caregiver to expose the opening. The caregiver may then apply an indicator assembly to the drape layer over the opening so that the indicator assembly receives fluid from the wound through the wicking structure. The indicator can include a portion that is responsive to fluid to provide a visual indication to the caregiver regarding a healing progression of the wound and/or to provide a visual indication of whether or not the wound is infected.

### Wound Dressing

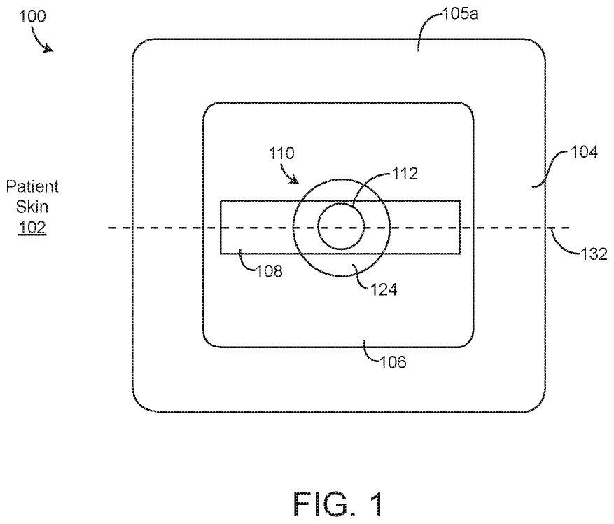

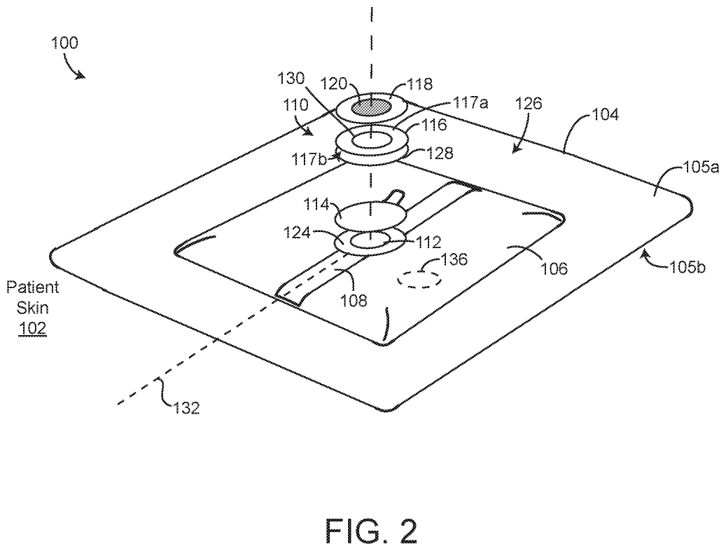

Referring particularly to FIGS. 1 and 2, a dressing 100 includes a drape, a drape layer, a barrier layer, a film layer, etc., shown as wound drape 104, according to some embodiments. Dressing 100 can also an absorbent core, a foam core, a body layer, a main body, a core structure, a spongy material, a compressible material, etc., shown as absorbent core 106. Dressing 100 can also include a wicking structure, a wicking member, a hydrophilic material, an absorbent layer or structure, etc., shown as wicking member 108. Wicking member 108 can be a wicking strip, a wicking structure, an absorbent structure, etc., and may have a hydrophilicity that is greater than a hydrophilicity of the absorbent core 106.

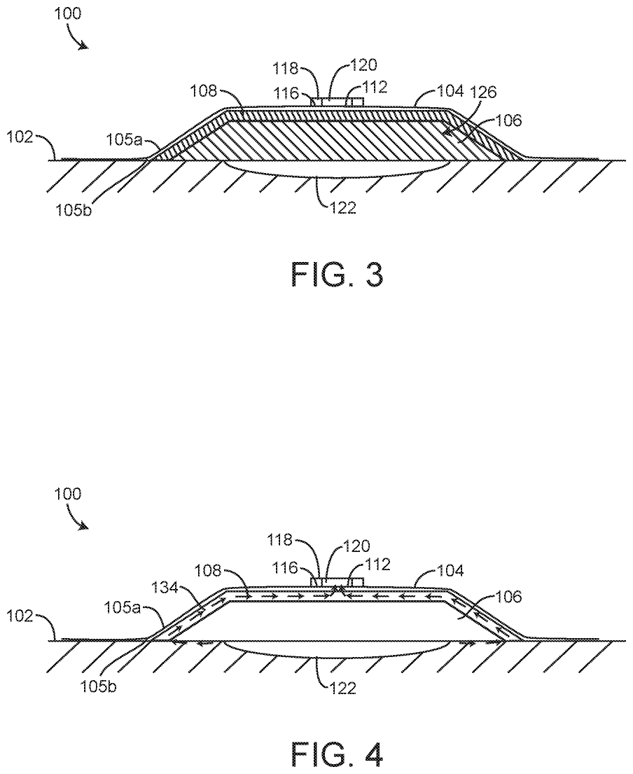

Wound drape 104 can be a polyurethane (PU) film layer that extends over a patient's wound 122 (shown in FIGS. 3 and 4). In some embodiments, wound drape 104 includes an exterior surface, an exterior side, an environment facing surface, an outwards facing surface, etc., shown as exterior surface 105a and an interior surface, an interior side, a wound facing surface, a wound facing side, an inwards facing surface, etc., shown as interior surface 105b. Exterior surface 105a and interior surface 105b may be opposite sides of wound drape 104. Wound drape 104 can extend over the patient's wound 122 and may sealingly couple with the patient's periwound skin 102. Wound drape 104 may sealingly couple circumferentially or peripherally about the patient's wound 122 at skin 102. Wound drape 104 can include an adhesive on interior surface 105b that is configured to facilitate sealed coupling or attachment between wound drape 104 and the patient's skin 102. Wound drape 104 may seal with the patient's skin 102 surrounding the patient's wound 122 to define an inner volume, a space, an internal volume, etc., shown as inner volume 126 in FIGS. 3 and 4. In other embodiments, wound drape 104 does not include the adhesive along the interior surface 105b of wound drape 104 and is otherwise sealingly coupled with the patient's skin 102. In some embodiments, the interior surface 105b of wound drape 104 includes the adhesive along portions or at different perimeters to facilitate sealingly coupling wound drape 104 with the patient's skin 102.

In some embodiments, absorbent core 106 and wicking member 108 are positioned within inner volume 126 of dressing 100. Absorbent core 106 can be configured to directly engage, contact, abut, etc., the patient's wound 122 to facilitate absorbing or removing fluid (e.g., wound fluid, wound exudate, etc.) from the patient's wound 122. Absorbent core 106 may be entirely positioned within inner volume 126. Inner volume 126 may be a sealed inner volume so that wound fluid that is absorbed by absorbent core 106 is retained within inner volume 126 and is prevented or restricted from leaking out of inner volume 126. In other embodiments, dressing 100 includes a wound interface layer such as a fenestrated PU film layer. The wound interface layer may be configured to directly engage the wound 122 and may be positioned between the wound 122 and the absorbent core 106.

In some embodiments, wicking member 108 is also entirely positioned within inner volume 126. Wicking member 108 may abut, directly contact, engage, etc., the interior surface 105b of wound drape 104. Wicking member 108 can abut or engage or contact the interior surface 105b of wound drape 104 on one side and abut or engage absorbent core 106 on an opposite or interior or wound-facing side. Wicking member 108 may extend laterally along an exterior or outwards facing surface of absorbent core 106. In some embodiments, wicking member 108 extends between wound drape 104 and absorbent core 106 but is still positioned within inner volume 126 that is defined by wound drape 104. Wicking member 108 may extend laterally along the exterior surface of absorbent core 106 at a center point of absorbent core 106. In other embodiments, wicking member 108 extends laterally along the exterior surface of absorbent core 106 at a position that is offset from the center point of absorbent core 106. In still other embodiments, multiple wicking members 108 are used that extend in one or more lateral directions along absorbent core 106.

Wicking members 108 are intended, arranged, or configured to provide a wound fluid passage or pathway to direct wound fluid to an optional indicator (e.g., indicator assembly 110). In some embodiments, wicking member 108 are pre-existing infrastructure of the dressing 100. For example, wicking member(s) 108 can be configured to wick, transfer, etc., wound fluid from wound 122 to indicator assembly 110 or a position of indicator assembly 110 even if the indicator is not currently used. For example, the indicator may be optionally installed, attached, positioned, disposed, etc., on the dressing 100 if desired by a caregiver. In this way, wicking member(s) 108 may be pre-existing infrastructure of the dressing 100 and the dressing 100 can be optionally converted between an “indicator” dressing (e.g., when dressing 100 includes indicator assembly 110) and a “non-indicator” dressing (e.g., when dressing 100 does not include indicator assembly 110). Specifically, wicking member(s) 108 may facilitate defining, producing, or creating a wound fluid passageway from wound 122 to an opening, port, or aperture on dressing 100 (e.g., aperture 112 as described in greater detail below) which may be releasably covered and can optionally receive a variety of indicators for providing a visual indication of wound healing progress.

For example, wicking member 108 may extend laterally along at least one outer periphery of absorbent core 106 along a lateral axis 132. In other embodiments, more than one wicking member 108 extend along other lateral axes that are offset from lateral axis 132 or are perpendicular with lateral axis 132.

In some embodiments, wicking member 108 is coupled with wound drape 104 through the adhesive that is coated along the interior surface 105b of wound drape 104. In other embodiments, if wound drape 104 does not include adhesive along the interior surface 105b, wicking member 108 may include an adhesive along an exterior surface or the surface of wicking member 108 that engages, contacts, abuts, etc., interior surface 105b of wound drape 104. In still other embodiments, wicking member 108 and wound drape 104 do not include adhesive and wicking member 108 is retained in place relative to wound drape 104 by engagement between wicking member 108 and wound drape 104.

Wound drape 104 can include an opening, a hole, an aperture, a perforation, an exit opening, a fluid opening, etc., shown as aperture 112. In some embodiments, aperture 112 extends through an entire thickness of wound drape 104. For example, aperture 112 may be an opening that is formed in wound drape 104 and extends between the interior surface 105b and the exterior surface 105a of wound drape 104. In some embodiments, aperture 112 fluidly couples inner volume 126 with an external environment of dressing 100. For example, aperture 112 may extend through wound drape 104 such that fluid may exit inner volume 126 through aperture 112.

Aperture 112 can be positioned on wound drape 104 such that aperture 112 at least partially overlaps wicking member 108. In some embodiments, aperture 112 is positioned on wound drape 104 so that aperture 112 completely overlaps wicking member 108. For example, aperture 112 may be positioned on wound drape 104 so that fluid (e.g., wound fluid, wound exudate, etc.) can exit inner volume 126 of dressing 100 through aperture 112. In some embodiments, aperture 112 is a circular hole having a diameter that is 10-15 millimeters. In some embodiments, aperture 112 aligns with wicking member 108 so that wicking member 108 can provide fluid to or through aperture 112. In some embodiments, wicking member 108 provides fluid to or through aperture 112 even when absorbent core 106 is not fully or partially saturated.

In some embodiments, aperture 112 is pre-manufactured in wound drape 104. For example, aperture 112 may be present when dressing 100 is provided to a user or to a customer. In other embodiments, aperture 112 is created by a nurse, a caregiver, etc., when applying dressing 100 to wound 122. For example, the caregiver may cut a hole in wound drape 104 to provide aperture 112. In this way, the caregiver may readily convert any dressing that includes the wicking member 108 to a dressing with a wound status indicator.

In some embodiments, dressing 100 includes a pull-tab, a removable cover, a film member, a removable film, etc., shown as film cover 114. Film cover 114 may be removably coupled or removably attached with the exterior surface 105a of wound drape 104. Film cover 114 may adhere to the exterior surface 105a of wound drape 104. For example, wound drape 104 may include a ring 124 of adhesive that surrounds (e.g., circumferentially, peripherally, etc.) aperture 112. In some embodiments, film cover 114 covers aperture 112 such that when film cover 114 is removed, aperture 112 fluidly couples inner volume 126 with the external environment. Before film cover 114 is removed, film cover 114 may facilitate maintaining a sealed inner volume 126.

In some embodiments, removal of film cover 114 produces aperture 112. For example, film cover 114 may be adhered or attached to a portion of wound drape 104 that is perforated circumferentially. In some embodiments, removal of film cover 114 results in removal of the perforated portion of wound drape 104, thereby producing or providing aperture 112. Removal of film cover 114 may expose aperture 112 (e.g., if aperture 112 is already present in wound drape 104) or may produce aperture 112 (e.g., by tearing a perforated portion having a shape of aperture 112).

In some embodiments, if film cover 114 is adhered with the exterior surface 105a of wound drape 104, an adhesive bond strength between film cover 114 and wound drape 104 is approximately 50-60% of an adhesive bond strength between wound drape 104 and the patient's skin 102. Advantageously, this facilitates preventing unintentional removal of wound drape 104 from the patient's skin 102 when film cover 114 is removed. Advantageously, since the adhesive bond strength between film cover 114 and wound drape 104 is less than the adhesive bond strength between wound drape 104 and the patient's skin 102, removing film cover 114 will likely not result in removal of wound drape 104 from the patient's skin 102, or compromise of the seal between wound drape 104 and the patient's skin 102.

In some embodiments, film cover 114 is a PU film having a thickness of 50-100 microns. In some embodiments, film cover 114 is adhered with the exterior surface 105a of wound drape 104 using a PU gel adhesive. The adhesive may be positioned on the exterior surface 105a of wound drape 104 around aperture 112 or may be positioned on an under-side (e.g., a wound-facing or dressing-facing side) of film cover 114. In some embodiments, the adhesive has a thickness of 80-100 grams per square meter to ensure a seal and conformability of film cover 114 if film cover 114 is not removed and dressing 100 is used without wound status indication.

In some embodiments, wicking member 108 is or includes a polyvinylidene fluoride, polyvinylidene difluoride, non-woven fabric, sintered polymer sheet/foam, etc., material. Wicking member 108 can be any highly or suitably hydrophilic material such that fluid preferentially wicks along wicking member 108 before being absorbed into absorbent core 106. For example, fluid may be absorbed through or along wicking member 108 more quickly than absorbent core 106. In some embodiments, wicking member 108 has a hydrophilicity that is greater than a hydrophilicity of the absorbent core 106 so that fluid tends to wick along wicking member 108 before absorbent core 106. In some embodiments, wicking member 108 quickly establishes fluid communication between the wound and the indicator and operates during the remaining life of dressing as wicking member 108 absorbs wound fluid.

Referring still to FIGS. 1 and 2, dressing 100 can also include an indicator assembly, an optional wound status indicator, etc., shown as indicator assembly 110. In some embodiments, indicator assembly 110 is selectably or optionally coupled, attached, etc., with dressing 100, or more particularly with wound drape 104. For example, indicator assembly 110 may be adhered to the exterior surface 105a of wound drape 104. In some embodiments, indicator assembly 110 is placed or coupled with wound drape 104 along the exterior surface 105a of wound drape 104 at aperture 112. Alternatively, indicator assembly 110 can be included separately in dressing packaging (e.g., as a kit) for use at discretion of a caregiver. Indicator assembly 110 and dressing 100 may be provided in a sterilized or sealed package.

Indicator assembly 110 can include an attachment ring, an adhesive ring, etc., shown as adhesive ring 116 and an indicator, an indicator member, an indicator patch, etc., shown as indicator 118. In some embodiments, adhesive ring 116 is a PU film and may have a thickness of 50 microns. In some embodiments, indicator 118 is a PU film with a thickness of 50 microns. In some embodiments, indicator assembly 110 has an overall height that is negligible such that indicator 118 can engage, contact, abut, etc., wicking member 108.

In some embodiments, adhesive ring 116 is used to adhere or removably couple indicator 118 with wound drape 104. Adhesive ring 116 can include an interior side, an interior surface, a wound-facing surface, a wound-facing side, a dressing-facing surface, etc., shown as interior surface 117b and an exterior side, an exterior surface, an environment-facing surface, an environment-facing side, etc., shown as exterior surface 117a. In some embodiments, exterior surface 117a has an acrylic adhesive that is configured to form a strong and permanent bond between adhesive ring 116 and indicator 118. In some embodiments, the interior surface 117b of adhesive ring 116 includes a soft and/or conformable PU or other gel adhesive with a bond strength that is 50-60% of the adhesive bond strength between wound drape 104 and the patient's skin 102.

In some embodiments, indicator assembly 110 includes or is laminated with a liner, a cover, a protective release liner, etc., shown as removable liner 128. Removable liner 128 can be adhered to the interior surface 117b of adhesive ring 116 and may be removed by a caregiver to expose the adhesive on interior surface 117b of adhesive ring 116. In some embodiments, removable liner 128 protects the interior surface 117b of adhesive ring 116 before indicator assembly 110 is attached with wound drape 104. The user may remove removable liner 128 to expose the adhesive on interior surface 117b, and place adhesive ring 116 over aperture 112.

In some embodiments, adhesive ring 116 is a ring member having a central aperture, a central opening, a central hole, etc., shown as central opening 130. Central opening 130 may have a diameter or radius that is substantially equal to the diameter or radius of aperture 112. In some embodiments, central opening 130 facilitates the transfer of fluid from the inner volume 126 of dressing 100 to indicator 118.

Indicator 118 may be adhered or attached with adhesive ring 116 on the exterior surface 117a of adhesive ring 116. As described above, the exterior surface 117a of adhesive ring 116 may be at least partially coated with an adhesive to facilitate attaching indicator with adhesive ring 116.

In some embodiments, indicator 118 includes a responsive portion 120 that is configured to change color, opacity, shade, etc., in response to contacting fluid. The fluid can be transferred from the wound 122 to the responsive portion 120 through a pathway including the wicking member 108, aperture 112, and central opening 130. In some embodiments, responsive portion 120 is viewable from the external environment of dressing 100. For example, responsive portion 120 may be viewable from outside dressing 100 so a caregiver or user is provided with a visual indication of the status of wound 122 without requiring removal of dressing 100. In this way, indicator 118 may provide a visual indication to the caregiver or user even when dressing 100 is applied and remains undisturbed. In some embodiments, for example, indicator 118 may change color or opacity in response to a presence of a particular type of matter in the fluid exuded by the wound. For example, if the wound is infected, indicator 118 may change color to provide a visual indication to the caregiver or the user that the wound is infected.

In some embodiments, responsive portion 120 is an ink that is responsive to fluid, wound exudate, etc. Responsive portion 120 may be printed onto indicator 118. In some embodiments, responsive portion 120 is printed onto indicator 118 and indicator 118 is a hydrophilic material.

Referring still to FIGS. 1 and 2, wicking member 108 may also function as a bacterial guard against port-based infection. Wicking member 108 can facilitate ensuring that fluid is always wicked in a direction away from the wound. In some embodiments, wicking member 108 is manufactured from polyvinylidene difluoride to facilitate wicking of fluid from wound 122 to indicator 118. In some embodiments, wicking member 108 entirely covers or overlaps the aperture 112 to facilitate preventing absorbent core 106 from being exposed to the external environment.

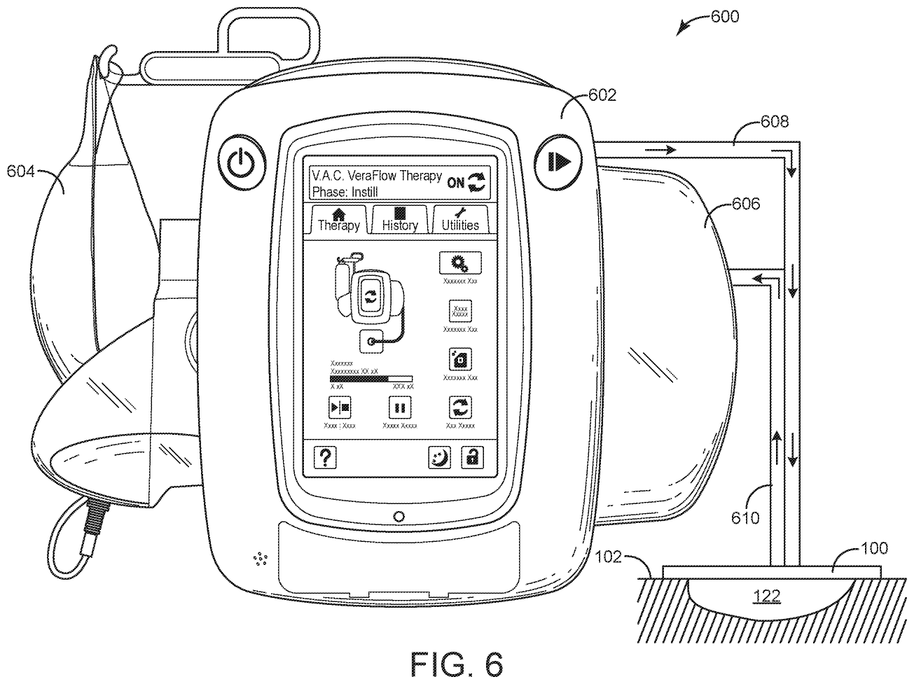

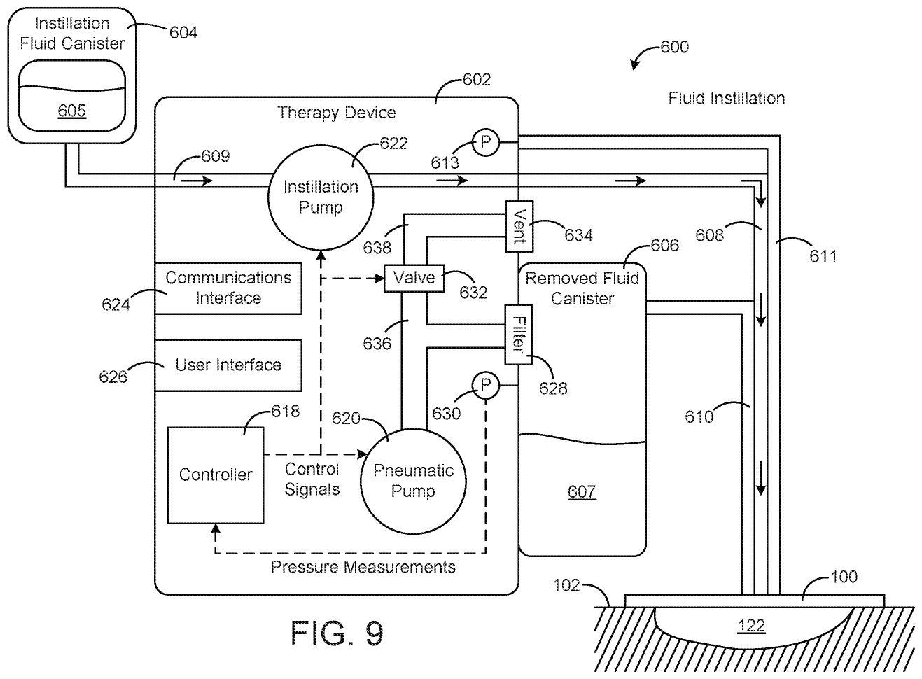

In some embodiments when dressing 100 is used in NPWT application, dressing 100 includes a negative pressure port, a valve, a vent, an opening, etc., shown as negative pressure port 136. For example, dressing 100 may be configured for use with a negative pressure unit (e.g., therapy device 602 as described in greater detail below with reference to FIGS. 6-9) and may fluidly couple with the negative pressure unit or tubular members of the negative pressure unit at negative pressure port 136. In some embodiments, the negative pressure port 136 is configured to fluidly couple the inner volume 126 with the negative pressure unit so that the negative pressure unit can draw a negative pressure within dressing 100 to facilitate healing progression of wound 122. In some embodiments, negative pressure port 136 is an opening that extends through wound drape 104 to inner volume 126.

Referring particularly to FIGS. 3-4, sectional views of dressing 100 are shown, according to some embodiments. FIG. 3 shows a sectional view of the construction of dressing 100 after dressing 100 has been fully configured for wound status indication. FIG. 4 shows a sectional view of dressing 100 demonstrating fluid flow paths.

As shown in FIG. 3, absorbent core 106 may be placed onto wound 122. In some embodiments, absorbent core 106 directly engages, contacts, abuts, etc., wound 122. Absorbent core 106 may have a coverage area that is substantially equal to or greater than an area of wound 122. Absorbent core 106 is positioned within inner volume 126 of dressing 100 which is defined by wound drape 104. Wound drape 104 may extend over absorbent core 106 with wicking member 108 positioned between wound drape 104 and absorbent core 106. Wound drape 104 sealingly couples (e.g., with an adhesive) with the patient's skin 102 surrounding wound 122 so that a sealed inner volume (i.e., inner volume 126) is defined.

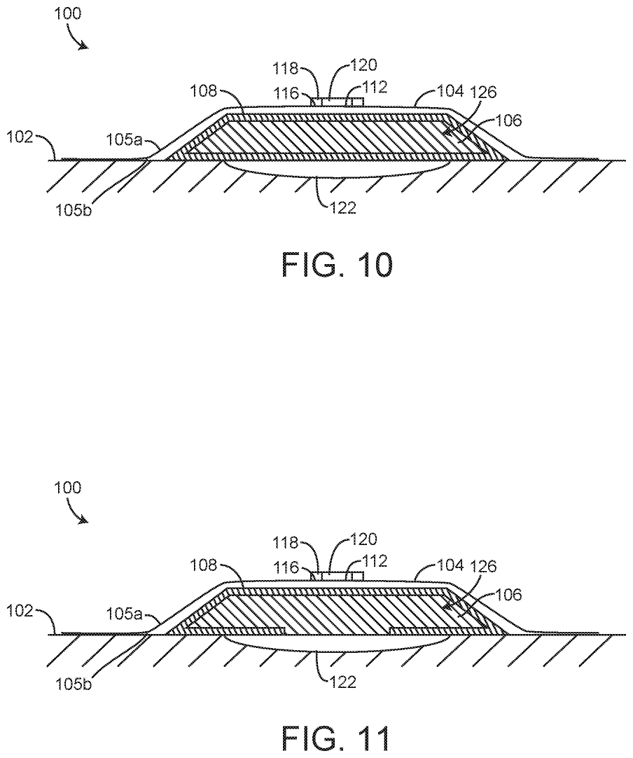

Referring particularly to FIG. 4, wicking member 108 may at least partially define a fluid pathway 134. Fluid pathway 134 extends from wound 122, around absorbent core 106, along wicking member 108, through aperture 112, and to indicator 118. In some embodiments, wicking member 108 extends along an exterior surface of absorbent core 106 to the patient's skin 102. In some embodiments, wicking member 108 also extends at least partially between the patient's skin 102 and absorbent core 106 as shown in FIGS. 10-11. For example, wicking member 108 may extend across wound 122 between absorbent core 106 and skin 102 (shown in FIG. 10) or may extend partially across or up to wound 122 (shown in FIG. 11).

Referring still to FIG. 4, wicking member 108 facilitates the transfer, transport, absorption, wicking, etc., of fluid exuded by wound 122 along fluid pathway 134 to indicator 118. Wicking member 108 absorbs the fluid from wound 122 and facilitates the transport of fluid along fluid pathway 134. In some embodiments, wicking member 108 defines fluid pathway 134 along an outer periphery or an outer surface of absorbent core 106. Wicking member 108 may directly contact, engage, abut, etc., indicator 118. In some embodiments, wicking member 108 directly contacts, engages, abuts, etc., the responsive portion 120 of indicator 118 so that fluid that is absorbed, wicked, transferred, transported, etc., by wicking member 108 may contact or react with responsive portion 120 of indicator 118. In some embodiments, the fluid that is wicked through wicking member 108 is transferred to responsive portion 120 through aperture 112 and/or central opening 130 of adhesive ring 116.

Responsive portion 120 may be configured to change color, or opacity in response to the fluid wicked from wound 122 to indicator 118 along or through wicking member 108. For example, responsive portion 120 may be configured to change from a first color to a second color, or from opaque to transparent or vice versa, etc., in response to receiving fluid from wound 122 that includes bacteria. In some embodiments, responsive portion 120 is configured to provide a visual indication of whether or not wound 122 is infected (e.g., whether or not bacteria is present in the fluid exuded by wound 122) or to provide a visual indication of a progression of healing of wound 122. For example, responsive portion 120 may change color or opacity based on a type of fluid or based on a type of matter present (or a concentration of matter present) in the fluid exuded by wound 122 to indicate a healing status or healing progression of wound 122. Responsive portion 120 can function as a sensor, a detector, an indicator, etc., to provide a visual indication to the user or caregiver of whether or not wound 122 is infected (e.g., whether or not bacteria is present in the exuded wound fluid) or to provide a visual indication of a healing progression of wound 122. For example, as wound 122 heals and a concentration of a particular type of matter in the fluid decreases, responsive portion 120 may transition from a first color to a different color, a different shade of color, a different opacity, etc.

In some embodiments, indicator assembly 110 is optional. For example, dressing 100 may be used even without providing indicator assembly 110. Advantageously, when a caregiver wishes to test wound 122 to determine if wound 122 is infected or to determine a healing progression of wound 122, the caregiver may apply indicator assembly 110 to opening 130 and view responsive portion 120. The caregiver may also replace indicator assembly 110 with a different indicator assembly 110 to re-perform the test or conduct a different test. For example, the caregiver may apply different types of indicator assembly 110 with indicators 118 that change color or opacity to provide visual indications of wound healing, infection, etc. In some embodiments, indicator assembly 110 and dressing 100 function to draw the exuded wound fluid to indicator 118 so that the caregiver can view the exuded wound fluid to determine if wound 122 is infected or to determine a healing progression of wound 122. For example, responsive portion 120 may be replaced with a transparent or translucent film so that the fluid wicked by wicking member 108 is viewable by the caregiver through wound drape 104.

In some embodiments, indicator assembly 110 is removable and replaceable after applied to dressing 100. For example, the caregiver may remove indicator assembly 110 and replace indicator assembly 110 with another indicator assembly 110 to re-perform the test.

In some embodiments, dressing 100 can include a soft polymer adapter that is adhered proximate aperture 112. In some embodiments, the soft polymer adapter accepts a test strip via a wipe valve so that the user or caregiver may insert a larger test strip and test more measurands and/or assays. In some embodiments, the soft polymer adapter and/or the aperture 112 is in fluid connection with wicking member 108 via a valve (e.g., a one-way valve) so that the user or caregiver or a therapy unit may dose fluids to inner volume 126 and wound 122 to reduce bacteria and/or to change wound conditions to facilitate wound healing. In some embodiments, aperture 112 and/or a separate opening are configured to accommodate or receive a syringe nozzle.

Advantageously, dressing 100 may be optionally converted from a dressing without wound status indication into a dressing with wound status indication. Dressing 100 is a versatile dressing that can be converted for wound status indication and may be re-used with different indicators. Dressing 100 can also be advantageously configurable for different test strips, wound indicators, etc. For example, indicator assembly 110 may be removed and replaced multiple times over time to facilitate wound status indication or healing progression without requiring removal of dressing 100. Dressing 100 is capable of optionally receiving a diagnostic patch (e.g., indicator 118 and/or indicator assembly 110). Additionally, dressing 100 can accommodate a range of indicators which may be selected as appropriate by the user or the caregiver without the need for a different type of dressing for the various indicators. Further, dressing 100 provides the benefits of dressing 100 in addition to the benefits of indicator assembly 110 without requiring manufacturing of both dressing 100 and indicator assembly 110 together.

### Process

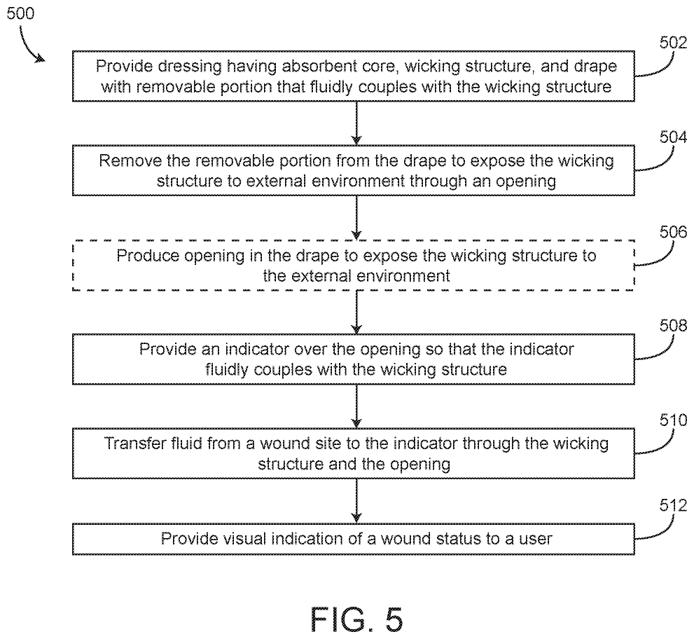

Referring particularly to FIG. 5, a process 500 for applying and using the dressing 100 as described in greater detail with reference to FIGS. 1-4 is shown, according to some embodiments. Process 500 includes steps 502-510 and can be performed to convert dressing 100 to a dressing with wound status indication. In some embodiments, process 500 can be performed for any dressing including a wicking member 108 that can be converted to a dressing with wound status indication, or for dressing 100 that is pre-configured to be converted for wound status indication.

Process 500 includes providing a dressing having an absorbent core, a wicking structure, and a drape (step 502), according to some embodiments. In some embodiments, the drape has a removable portion that can be removed to define an aperture or opening in the drape to fluidly couple an interior volume of the dressing with exterior or external environment. For example, the drape may be wound drape 104 including film cover 114 that can be removed from wound drape 104 to expose aperture 112 which is positioned or aligned over wicking member 108, thereby fluidly coupling inner volume 126 with the environment that is external to dressing 100. In other embodiments, the drape does not include a removable portion, and aperture 112 is produced by making an incision in the drape at the wicking member. The dressing may be provided over a patient's wound and may sealingly couple (e.g., the drape may sealingly couple) with the patient's skin surrounding the wound. In some embodiments, the drape adheres to the patient's skin to define the inner volume of the dressing over the wound.

Process 500 includes removing the removable portion from the drape to expose the wicking structure to the external environment through an opening (step 504), according to some embodiments. In some embodiments, step 504 is performed by a technician, a nurse, a caregiver, etc., after the dressing has been applied to the wound. For example, step 504 can be performed by pulling or removing film cover 114 from wound drape 104. In some embodiments, film cover 114 is a perforated portion of wound drape 104 such that removal of film cover 114 produces aperture 112. In other embodiments, film cover 114 is a member that is adhered to the exterior surface of wound drape 104 and removal of film cover 114 exposes aperture 112 which is already formed in wound drape 104. Aperture 112 may be aligned on wicking member 108 or otherwise positioned such that aperture 112 fluidly couples the inner volume of the dressing with the external environment through wicking member 108.

Process 500 optionally includes producing an opening in the drape to expose the wicking structure to the external environment (step 506), according to some embodiments. In some embodiments, step 506 is performed if the dressing does not include the removable portion. For example, if the drape is continuous and does not include a perforated portion or a removable portion for producing or exposing the opening, step 506 may be performed. In some embodiments, step 506 is performed by a caregiver after or before the dressing is applied to the patient's wound. The caregiver may make an incision in the drape at the wicking structure to expose the wicking structure to the external environment through the incision (i.e., the opening).

Process 500 includes providing an indicator over the opening so that the indicator fluidly couples with the wicking structure (step 508), according to some embodiments. In some embodiments, the indicator sealingly couples with the exterior surface of the drape so that fluid can be provided to the indicator through the wicking structure and the opening, but is prevented or restricted from leaking through the sealed coupling between the indicator and the exterior surface of the drape.

Process 500 includes transferring fluid from a wound site, a wound bed, or the wound, to the indicator through the wicking structure and the opening (step 510), according to some embodiments. In some embodiments, the wicking structure is configured to wrap around the absorbent core to the wound site. The wicking structure can be more absorbent than the absorbent core so that fluid preferentially is absorbed, wicked, transferred, etc., through the wicking structure. In some embodiments, the wicking structure has a higher hydrophilicity than a hydrophilicity of the absorbent core. The wicking structure can be a laterally extending member that fluidly couples with both the opening in the drape and the wound site. In some embodiments, the wicking structure absorbs wound fluid faster, more rapidly, etc., than the absorbent core. In this way, the wicking structure may absorb, transfer, transport, wick, etc., wound fluid from the wound to the opening in the drape faster than fluid is absorbed into the absorbent core. In some embodiments, the wicking structure provides a rapid fluid delivery pathway from the wound to the opening (or the indicator) even when the absorbent core is only partially saturated, not saturated at all, or fully saturated.

Process 500 includes providing a visual indication of a wound status to a user (step 512), according to some embodiments. In some embodiments, step 512 is performed by the indicator. For example, the indicator may be manufactured from or include a material that is responsive to fluid. For example, the indicator may change color, opacity, etc., in response to contact with fluid that is exuded from the wound site and wicked to the indicator through the wicking structure. In some embodiments, the indicator may change color or opacity, or otherwise visually change in response to a presence of a particular type of matter in the fluid exuded by the wound. For example, the indicator may change color or opacity to provide a visual indication of infection of the wound. In some embodiments, the indicator is a test indicator that is configured to provide a visual indication of a status of the wound. The indicator may transition between different colors, different shades of a color, varying degrees of opacity, etc., in response to the fluid exuded from the wound and transferred to the indicator through the wicking structure. In this way, the indicator is viewable by a user without removal or disturbance of the dressing.

### Wound Therapy System

Referring now to FIGS. 6-9, a negative pressure wound therapy (NPWT) system 600 is shown, according to an exemplary embodiment. NPWT system 600 is shown to include a therapy device 602 fluidly connected to a wound dressing 100 via tubing 608 and 610. Wound dressing 100 may be adhered or sealed to a patient's skin 102 surrounding a wound 122. Several examples of wound dressings 100 which can be used in combination with NPWT system 600 are described in detail in U.S. Pat. No. 7,651,484 granted Jan. 26, 2010, U.S. Pat. No. 8,394,081 granted Mar. 12, 2013, and U.S. patent application Ser. No. 14/087,418 filed Nov. 22, 2013. The entire disclosure of each of these patents and patent applications is incorporated by reference herein.

Therapy device 602 can be configured to provide negative pressure wound therapy by reducing the pressure at wound 122. Therapy device 602 can draw a vacuum at wound 122 (relative to atmospheric pressure) by removing wound exudate, air, and other fluids from wound 122. Wound exudate may include fluid that filters from a patient's circulatory system into lesions or areas of inflammation. For example, wound exudate may include water and dissolved solutes such as blood, plasma proteins, white blood cells, platelets, and red blood cells. Other fluids removed from wound 122 may include instillation fluid 605 previously delivered to wound 122. Instillation fluid 605 can include, for example, a cleansing fluid, a prescribed fluid, a medicated fluid, an antibiotic fluid, or any other type of fluid which can be delivered to wound 122 during wound treatment. Instillation fluid 605 may be held in an instillation fluid canister 604 and controllably dispensed to wound 122 via instillation fluid tubing 608. In some embodiments, instillation fluid canister 604 is detachable from therapy device 602 to allow canister 606 to be refilled and replaced as needed.

The fluids 607 removed from wound 122 pass through removed fluid tubing 610 and are collected in removed fluid canister 606. Removed fluid canister 606 may be a component of therapy device 602 configured to collect wound exudate and other fluids 607 removed from wound 122. In some embodiments, removed fluid canister 606 is detachable from therapy device 602 to allow canister 606 to be emptied and replaced as needed. A lower portion of canister 606 may be filled with wound exudate and other fluids 607 removed from wound 122, whereas an upper portion of canister 606 may be filled with air. Therapy device 602 can be configured to draw a vacuum within canister 606 by pumping air out of canister 606. The reduced pressure within canister 606 can be translated to wound dressing 100 and wound 122 via tubing 610 such that wound dressing 100 and wound 122 are maintained at the same pressure as canister 606.

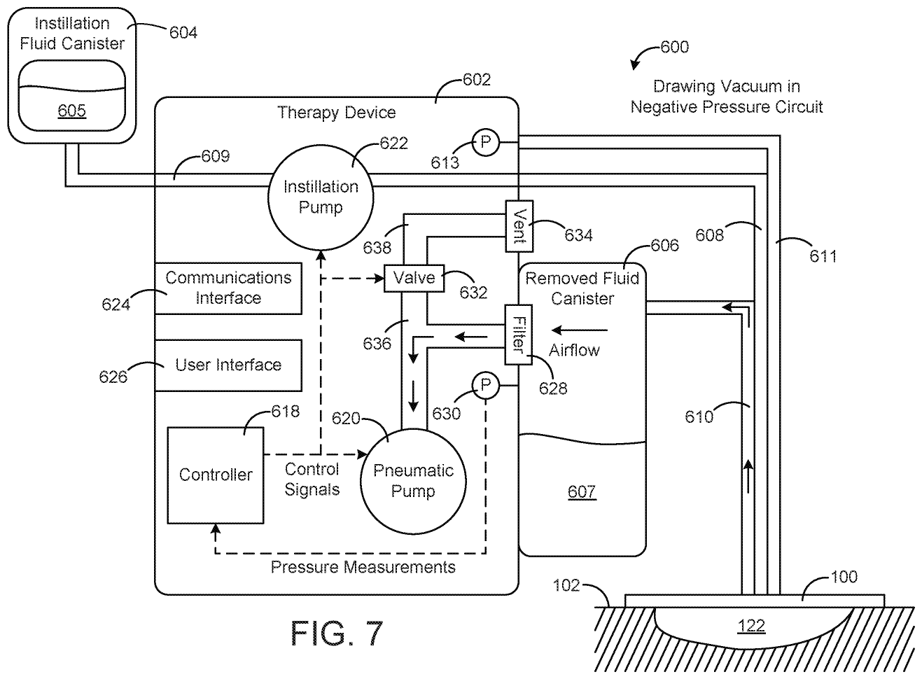

Referring particularly to FIGS. 7-9, block diagrams illustrating therapy device 602 in greater detail are shown, according to an exemplary embodiment. Therapy device 602 is shown to include a pneumatic pump 620, an instillation pump 622, a valve 632, a filter 628, and a controller 618. Pneumatic pump 620 can be fluidly coupled to removed fluid canister 606 (e.g., via conduit 636) and can be configured to draw a vacuum within canister 606 by pumping air out of canister 606. In some embodiments, pneumatic pump 620 is configured to operate in both a forward direction and a reverse direction. For example, pneumatic pump 620 can operate in the forward direction to pump air out of canister 606 and decrease the pressure within canister 606. Pneumatic pump 620 can operate in the reverse direction to pump air into canister 606 and increase the pressure within canister 606. Pneumatic pump 620 can be controlled by controller 618, described in greater detail below.

Similarly, instillation pump 622 can be fluidly coupled to instillation fluid canister 604 via tubing 609 and fluidly coupled to wound dressing 100 via tubing 608. Instillation pump 622 can be operated to deliver instillation fluid 605 to wound dressing 100 and wound 122 by pumping instillation fluid 605 through tubing 609 and tubing 608, as shown in FIG. 4. Instillation pump 622 can be controlled by controller 618, described in greater detail below.

Filter 628 can be positioned between removed fluid canister 606 and pneumatic pump 620 (e.g., along conduit 636) such that the air pumped out of canister 606 passes through filter 628. Filter 628 can be configured to prevent liquid or solid particles from entering conduit 636 and reaching pneumatic pump 620. Filter 628 may include, for example, a bacterial filter that is hydrophobic and/or lipophilic such that aqueous and/or oily liquids will bead on the surface of filter 628. Pneumatic pump 620 can be configured to provide sufficient airflow through filter 628 that the pressure drop across filter 628 is not substantial (e.g., such that the pressure drop will not substantially interfere with the application of negative pressure to wound 122 from therapy device 602).

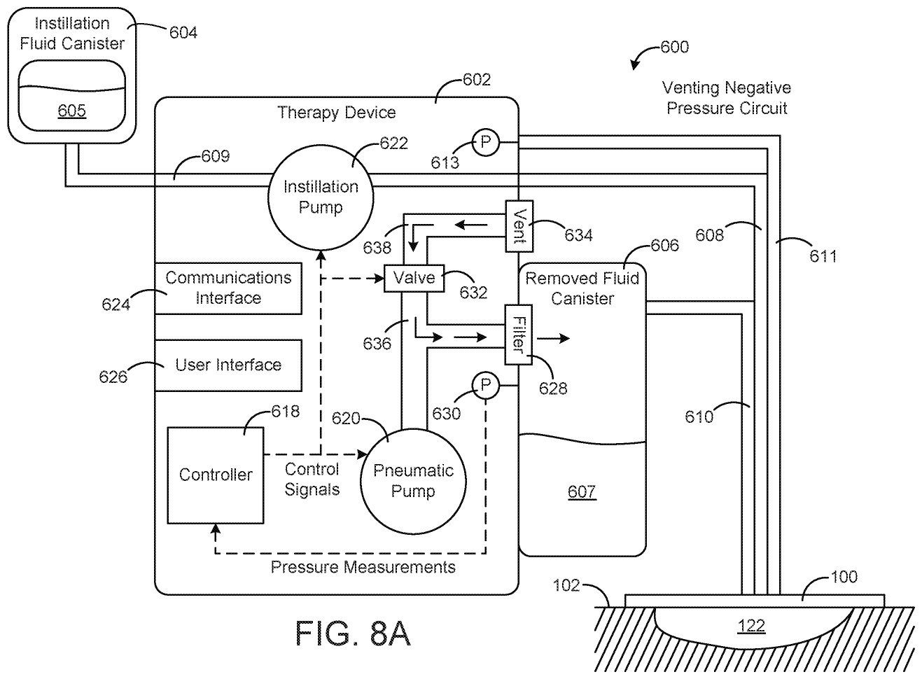

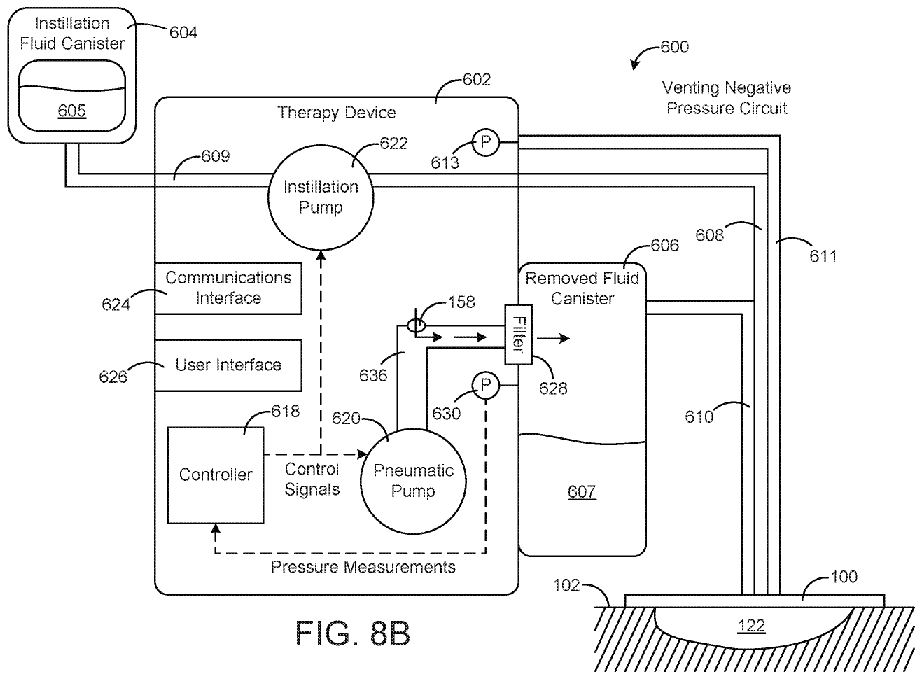

In some embodiments, therapy device 602 operates a valve 632 to controllably vent the negative pressure circuit, as shown in FIG. 8A. Valve 632 can be fluidly connected with pneumatic pump 620 and filter 628 via conduit 636. In some embodiments, valve 632 is configured to control airflow between conduit 636 and the environment around therapy device 602. For example, valve 632 can be opened to allow airflow into conduit 636 via vent 634 and conduit 638, and closed to prevent airflow into conduit 636 via vent 634 and conduit 638. Valve 632 can be opened and closed by controller 618, described in greater detail below. When valve 632 is closed, pneumatic pump 620 can draw a vacuum within a negative pressure circuit by causing airflow through filter 628 in a first direction, as shown in FIG. 7. The negative pressure circuit may include any component of system 600 that can be maintained at a negative pressure when performing negative pressure wound therapy (e.g., conduit 636, removed fluid canister 606, tubing 610, wound dressing 100, and/or wound 122). For example, the negative pressure circuit may include conduit 636, removed fluid canister 606, tubing 610, wound dressing 100, and/or wound 122. When valve 632 is open, airflow from the environment around therapy device 602 may enter conduit 636 via vent 634 and conduit 638 and fill the vacuum within the negative pressure circuit. The airflow from conduit 636 into canister 606 and other volumes within the negative pressure circuit may pass through filter 628 in a second direction, opposite the first direction, as shown in FIG. 8A.

In some embodiments, therapy device 602 vents the negative pressure circuit via an orifice 158, as shown in FIG. 8B. Orifice 158 may be a small opening in conduit 636 or any other component of the negative pressure circuit (e.g., removed fluid canister 606, tubing 610, tubing 611, wound dressing 100, etc.) and may allow air to leak into the negative pressure circuit at a known rate. In some embodiments, therapy device 602 vents the negative pressure circuit via orifice 158 rather than operating valve 632. Valve 632 can be omitted from therapy device 602 for any embodiment in which orifice 158 is included. The rate at which air leaks into the negative pressure circuit via orifice 158 may be substantially constant or may vary as a function of the negative pressure, depending on the geometry of orifice 158.

In some embodiments, therapy device 602 includes a variety of sensors. For example, therapy device 602 is shown to include a pressure sensor 630 configured to measure the pressure within canister 606 and/or the pressure at wound dressing 100 or wound 122. In some embodiments, therapy device 602 includes a pressure sensor 613 configured to measure the pressure within tubing 611. Tubing 611 may be connected to wound dressing 100 and may be dedicated to measuring the pressure at wound dressing 100 or wound 122 without having a secondary function such as channeling installation fluid 605 or wound exudate. In various embodiments, tubing 608, 610, and 611 may be physically separate tubes or separate lumens within a single tube that connects therapy device 602 to wound dressing 100. Accordingly, tubing 610 may be described as a negative pressure lumen that functions apply negative pressure wound dressing 100 or wound 122, whereas tubing 611 may be described as a sensing lumen configured to sense the pressure at wound dressing 100 or wound 122. Pressure sensors 630 and 613 can be located within therapy device 602, positioned at any location along tubing 608, 610, and 611, or located at wound dressing 100 in various embodiments. Pressure measurements recorded by pressure sensors 630 and/or 613 can be communicated to controller 618. Controller 618 use the pressure measurements as inputs to various pressure testing operations and control operations performed by controller 618.

Controller 618 can be configured to operate pneumatic pump 620, instillation pump 622, valve 632, and/or other controllable components of therapy device 602. In some embodiments, controller 618 operates pneumatic pump 620, instillation pump 622, valve 632, and/or other controllable components of therapy device 602 to draw a negative pressure at wound 122 and/or to provide instillation fluid to wound 122.

In some embodiments, therapy device 602 includes a user interface 626. User interface 626 may include one or more buttons, dials, sliders, keys, or other input devices configured to receive input from a user. User interface 626 may also include one or more display devices (e.g., LEDs, LCD displays, etc.), speakers, tactile feedback devices, or other output devices configured to provide information to a user. In some embodiments, the pressure measurements recorded by pressure sensors 630 and/or 613 are presented to a user via user interface 626. User interface 626 can also display alerts generated by controller 618. For example, controller 618 can generate a “no canister” alert if canister 606 is not detected.

In some embodiments, therapy device 602 includes a data communications interface 624 (e.g., a USB port, a wireless transceiver, etc.) configured to receive and transmit data. Communications interface 624 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, communications interface 624 can include a USB port or an Ethernet card and port for sending and receiving data via an Ethernet-based communications link or network. In another example, communications interface 624 can include a Wi-Fi transceiver for communicating via a wireless communications network or cellular or mobile phone communications transceivers.

### CONFIGURATION OF EXEMPLARY EMBODIMENTS

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, and/or illustrations of possible embodiments (and such terms are not intended to connote that such embodiments are necessarily extraordinary or superlative examples).

The term “coupled,” as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. Such members may be coupled mechanically, electrically, and/or fluidly.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

The hydrophobicity of a material may vary, but to be considered hydrophobic, generally the material can have an average contact angle with water of at least 90 degrees in some embodiments. To be considered hydrophilic, generally the material can have a contact angle of most 90 degrees in some embodiments. In some embodiments the contact angle with water can be no more than 150 degrees. For example, in some embodiments, the contact angle of the hydrophobic material may be in a range of at least 70 degrees to about 120 degrees with an average contact angle of at least 90 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of a material herein may be further enhanced with a hydrophobic coating of other materials, such as silicones and fluorocarbons, and by any means known, such as by coating by the other material (e.g., coated using a liquid that may be subsequently dried on the material) or plasma coated.

A hydrophobic material can be any material having a solubility in water of less than 10 mg/L at standard temperature and pressure. A hydrophilic material can be any material having a solubility in water of 10 mg/L and greater at standard temperature and pressure.

It is important to note that the construction and arrangement of the wound dressing with optional status indicator as shown in the various exemplary embodiments is illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. Other substitutions, modifications, changes, and omissions may be made in the design, operating conditions and arrangement of the exemplary embodiments without departing from the scope of the present disclosure.

Additionally, any element disclosed in one embodiment may be incorporated or utilized with any other embodiment disclosed herein. For example, the configuration and construction of dressing 10 of the exemplary embodiment described in at least paragraph [0067] may be incorporated in the dressing 10 of the exemplary embodiment described in at least paragraph [0036]. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A dressing for a wound comprising:
a drape configured to sealingly couple with a patient's skin surrounding the wound, wherein the drape encloses an inner volume of the dressing;
an absorbent core layer positioned within the inner volume of the dressing;
a wicking member positioned within the inner volume, wherein the wicking member extends from an underside of the absorbent core layer to the drape;
an indicator configured to fluidly couple with the wicking member through the drape and receive fluid from the wicking member, wherein the indicator is configured to provide a visual wound status indication in response to receiving fluid from the wound through the wicking member; and
an adhesive ring comprising:
an exterior surface configured to adhere to the indicator;
an interior surface configured to adhere to an exterior surface of the drape; and
a central opening aligned with the opening of the drape.

2. The dressing of claim 1, wherein the drape comprises an interior surface and an exterior surface, wherein the interior surface includes an adhesive configured to sealingly couple with the patient's skin surrounding the wound to define the inner volume.

3. The dressing of claim 1, wherein the drape comprises an opening aligned with the wicking member.

4. The dressing of claim 3, wherein the indicator is positioned at the opening and the wicking member defines a fluid flow path from the wound to the indicator through the opening in the drape.

5. The dressing of claim 1, wherein an adhesive bond strength between the indicator and the drape is less than an adhesive bond strength between the drape and the patient's skin.

6. The dressing of claim 5, wherein the adhesive bond strength between the indicator and the drape is 60% of the adhesive bond strength between the drape and the patient's skin or less than 60% of the adhesive bond strength between the drape and the patient's skin.

7. The dressing of claim 1, wherein the wicking member extends at least partially between the absorbent core layer and the patient's skin.

8. The dressing of claim 1, wherein the wicking member extends between the absorbent core layer and the patient's skin to the wound.

9. The dressing of claim 1, wherein a hydrophilicity of the wicking member is greater than a hydrophilicity of the absorbent core layer so that fluid exuded by the wound is absorbed and wicked through the wicking member to the indicator before being absorbed by the absorbent core layer.

10. The dressing of claim 1, wherein the dressing further comprises a negative pressure port configured to fluidly couple the inner volume of the dressing with a negative pressure unit.

11. The dressing of claim 10, wherein the negative pressure port is configured to receive tubing to fluidly couple the inner volume of the dressing with the negative pressure unit through the tubing.

12. The dressing of claim 1, wherein the dressing is configured to receive a plurality of different types of indicators for performing different tests.

13. A convertible dressing for a wound, the convertible dressing comprising:
an absorbent core;
a wicking member extending along an exterior surface of the absorbent core; and
a drape configured to extend over the absorbent core and the wicking member, wherein the drape comprises a removable portion configured to expose or produce an aperture in the drape, wherein the aperture is aligned with the wicking member.

14. The convertible dressing of claim 13, wherein the removable portion is a removable pull tab that is adhered with the drape over the aperture, wherein removal of the removable portion exposes the aperture.

15. The convertible dressing of claim 13, wherein the removable portion is a perforated portion of the drape, wherein removal of the removable portion produces the aperture in the drape.

16. The convertible dressing of claim 13, wherein the aperture is configured to receive an indicator for providing a visual wound indication.

17. The convertible dressing of claim 16, wherein a peripheral surface surrounding the aperture is configured to adhere with the indicator.

18. The convertible dressing of claim 17, wherein the peripheral surface surrounding the aperture is configured to adhere with a ring that adheres with the indicator, wherein the ring is positioned between the drape and the indicator.

19. A method of providing a dressing with a wound status indication, the method comprising:
providing a dressing having a wound drape that seals with a user's skin surrounding a wound, wherein the dressing comprises an absorbent core and a wicking member within the wound drape, wherein the wicking member extends between an underside of the absorbent core and the wound drape;
providing an opening in the wound drape aligned with the wicking member;
providing a visual indicator at the opening, wherein the visual indicator is configured to provide a visual wound status indication in response to receiving fluid from the wicking member.

20. The method of claim 19, wherein providing the opening in the wound drape comprises:
providing a perforated removable member, wherein removal of the perforated removable member produces the opening in the wound drape.

21. The method of claim 19, wherein providing the opening in the wound drape comprises:
providing a removable member adhered over the opening, wherein removal of the removable member exposes the opening in the wound drape.

22. The method of claim 19, wherein providing the opening in the wound drape comprises:
making an incision in the wound drape and removing a portion of the wound drape to produce the opening.

23. A kit for a wound dressing with optional status indication, the kit comprising:
a sealed package with sterilized interior containing at least:
a wound dressing having a drape, an absorbent core, and a wicking layer, wherein the drape comprises an opening and a removable cover on the opening; and
one or more wound status indicators configured to be optionally placed over the opening in communication with the wicking layer after removal of the removable cover.

24. The kit of claim 23, wherein the one or more wound status indicators are different types of indicators, each configured to provide a visual indication for a different test in response to contact with wound fluid.

25. The kit of claim 23, wherein the kit comprises a negative pressure port in the drape and tubing configured to fluidly couple an inner volume of the wound dressing with a therapy unit.
